(19) United States
(12) Patent Application Publication
(10) Pub. No.: US 2004/0030345 A1
(43) Pub. Date: Feb. 12, 2004

(54) Bone cement syringe

(76) Inventors: Gary Douglas Aurin, Foothill Ranch, CA (US); William M. Porter, Carlsbad, CA (US)

Correspondence Address: KLEIN, O'NEILL & SINGH, 2 Park Plaza, Ste. 510, Irvine, CA, 92614, US

(21) Appl. No.: 10/215,604

(22) Filed: Aug. 9, 2002

(51) Int. Cl. A61F 2/00

(52) U.S. Cl. 606/92

(57) ABSTRACT

An improved syringe for delivering a viscous liquid, especially bone cement, to a surgical site, has a barrel with a distal outlet end and an open proximal end, and a plunger movable axially within the barrel, the plunger having a shaft extending proximally from the proximal end of the barrel. The plunger shaft incorporates a reinforcing structure that provides increased torsional rigidity and improved dispersion of torsional loads, and the barrel includes an outwardly and proximally flared peripheral rim around its proximal end. The peripheral rim forms a funnel that circumscribes less than 360°, and preferably about 240° of the proximal end of the barrel, thereby defining a gap in the rim that provides direct access to the open proximal end of the barrel. The rim facilitates the filling of the barrel either by pouring a liquid into the funnel, or by filling the barrel with a highly viscous, paste-like liquid directly into the open proximal end of the barrel through the gap. The shaft is formed of a pair of flattened, elongate longitudinal members that intersect at right angles to form a shaft structure that is cruciform in cross section, wherein the reinforcing structure is provided by a truss element lattice formed between the longitudinal members.

### CROSS REFERENCE TO RELATED APPLICATIONS

This application is related to co-pending application Ser. No. 09/934,435; filed Aug. 21, 2001, the disclosure of which is incorporated herein by reference.

### FEDERALLY SPONSORED RESEARCH OR DEVELOPMENT

Not Applicable

### BACKGROUND OF THE INVENTION

The present invention relates to a syringe for delivering a viscous liquid material to a surgical site within the body of a human or an animal. More specifically, it relates to a syringe that is especially configured for use in combination with a syringe actuation device for controllably delivering bone cement to a site within a bone that has been surgically prepared to receive the cement.

Many procedures in orthopedic surgery require a predetermined quantity of bone cement to be delivered to a site within a bone that has been surgically prepared to receive the cement. For example, surgery to correct certain spinal injuries or deformities requires a hole to be drilled or bored in a vertebra, and then the hole is filled with bone cement. This is accomplished by filling a syringe with bone cement, and then delivering the cement to the site via a cannula attached to the syringe by a length of flexible tubing.

Because the cement may be quite thick and viscous, delivering the cement from the syringe often requires a great deal of effort applied to the syringe plunger. Thus, both strength and dexterity are required on the part of the surgeon performing the procedure.

Furthermore, the process of filling the syringe with bone cement has been troublesome in practice. Specifically, bone cement comes in varying consistencies and viscosities. Cement with a relatively low viscosity that is capable of liquid-like flow requires very accurate poring into the syringe to avoid spills, while high viscosity cement has a paste-like consistency, and thus must be forced down into the barrel of the syringe. In either case, a quick and accurate filling of the syringe may prove difficult, presenting possible complications due to the relatively quick setting time of the cement, and consequently a relatively narrow time interval for the delivery of the cement to the surgical site.

It would thus be an improvement over the current state of the art to provide a mechanism that would facilitate the delivery of bone cement and like materials by making it easier both to fill the syringe quickly and accurately with bone cements of varying consistencies and viscosities, and to express such materials from the syringe.

### SUMMARY OF THE INVENTION

Broadly, in one aspect, the present invention is a bone cement syringe having a barrel with a proximal end opening, and a flared peripheral rim around the proximal end opening. The rim flares proximally and radially outwardly, creating a funnel for the proximal opening. The flared peripheral rim circumscribes less than 360° of the proximal end opening, preferably about 210° to about 270°. This creates an interruption or gap in the funnel for direct access to the proximal opening. For bone cement of the less viscous type, the funnel allows quick and accurate pouring of a measured amount of the bone cement into the syringe barrel without spillage. For bone cement of the highly-viscous, paste-like type, the gap in the funnel allows the proximal opening to be directly accessed by a cement spatula or like instrument, so that the cement can be scraped off of the spatula into the opening.

The syringe of the present invention is designed for use with a syringe actuation device, of the type disclosed and claimed in prior co-pending U.S. application Ser. No. 09/934,435; filed Aug. 21, 2001, and commonly assigned to the assignee of the invention disclosed and claimed herein. The disclosure of the aforementioned application is expressly incorporated herein by reference. As disclosed in the prior application, the syringe actuation device comprises (1) a hollow, internally-threaded sleeve configured to receive the plunger in its withdrawn position, the sleeve having an open proximal end and a distal end opening configured for securing the syringe barrel; and (2) a substantially cylindrical syringe driving element having (a) an externally-threaded distal portion dimensioned to screw into the proximal end of the sleeve, and (b) a plunger seat, at the distal end of the driving element, that bears against the plunger and that pushes the plunger axially toward its inserted position in the barrel as the driving element is threaded into the sleeve.

In a specific embodiment disclosed in the above-referenced prior application, the actuation device comprises an internally-threaded hollow sleeve with an open proximal end, and a substantially cylindrical driving element with an externally-threaded distal portion that threads into the open proximal end of the sleeve. The driving element includes a transverse (i.e., perpendicular to the driving element axis) plunger seat at its distal end. The sleeve has a longitudinal slot parallel to its axis for receiving the extended plunger of a pre-filled syringe, and a distal portion with an opening through which the barrel of the syringe extends. As adapted for use with the present invention, the longitudinal slot is configured to receive the funnel-shaped peripheral rim of the syringe. Also, as disclosed in the above-referenced prior application, the proximal portion of the driving element is configured as an enlarged-diameter gripping element that facilitates actuation by increasing the mechanical advantage when the driving element is screwed into the sleeve.

In a preferred embodiment of the present invention, the syringe has a plunger that is optionally made with a reinforcing truss structure (as opposed to the simple cruciform structure of the prior art) that provides higher torsional rigidity and better dispersion of torsional loads as the plunger is pushed into the syringe barrel by the rotational screw action of the driving element. Furthermore, the plunger is advantageously formed with a radiused button or knob at its proximal end to minimize the transfer of torque to the plunger from the driving element.

In use, the syringe is filled with a predetermined amount of liquid, e.g., bone cement. The driving element is backed out of the sleeve a sufficient distance in the proximal direction to allow the filled syringe to be installed in the sleeve through the longitudinal opening. With the barrel of the syringe being filled with a measured volume of liquid, the plunger of the syringe is in its extended or withdrawn position. The outlet tip of the syringe is connected to one end of a fluid conduit, such as a length of flexible tubing, the other end of which may be coupled to an injection needle or a cannula. As the driving element is threaded into the sleeve, the plunger seat bears against the plunger to push the plunger distally into the barrel until it reaches its fully inserted position, corresponding to the delivery of the measured volume of liquid from the barrel.

It will be appreciated that the threaded coupling between the driving element and the sleeve allows the driving element to be turned as a screw within the sleeve and to advance against the plunger with the mechanical advantage provided by a screw mechanism. This screwing action, in turn, allows the user more easily to apply sufficient force to the plunger to express a highly viscous liquid (e.g., bone cement) from the barrel. Furthermore, a greater degree of control can be used in actuating the plunger. For example, stopping the plunger at precise positions within the barrel, so as to express the contents of the barrel in desired increments, is greatly facilitated. These and other advantages of the invention will be more fully understood from the detailed description that follows.

### BRIEF DESCRIPTION OF THE DRAWINGS

FIG. 1 is a perspective view of bone cement syringe, in accordance with a preferred embodiment of the present invention;

FIG. 2 is a perspective view of a syringe actuation device, of the type that may be used in conjunction with the preferred embodiment of the present invention;

FIG. 3 is a longitudinal cross-sectional view taken along line 3-3 of FIG. 2;

FIG. 4 is a cross-sectional view similar to that of FIG. 3, but showing the syringe of FIG. 1 (in elevation) installed in the syringe actuation device;

FIG. 5 is a cross-sectional view, similar to that of FIG. 4, but showing the proximal portion of the syringe in cross-section;

FIG. 6 is a cross-sectional view taken along line 6-6 of FIG. 5;

FIG. 7 is a cross-sectional view taken along line 7-7 of FIG. 5;

FIG. 8 is a cross-sectional view taken along line 8-8 of FIG. 5; and

FIGS. 9-11 are perspective views of a device for installing a cement cannula into a bone, showing the steps in the method of installing the cannula; and

FIG. 12 is a perspective view of a bone cement delivery apparatus incorporating the syringe of the present invention, connected to the cannula of FIGS. 9-11.

### DETAILED DESCRIPTION OF THE INVENTION

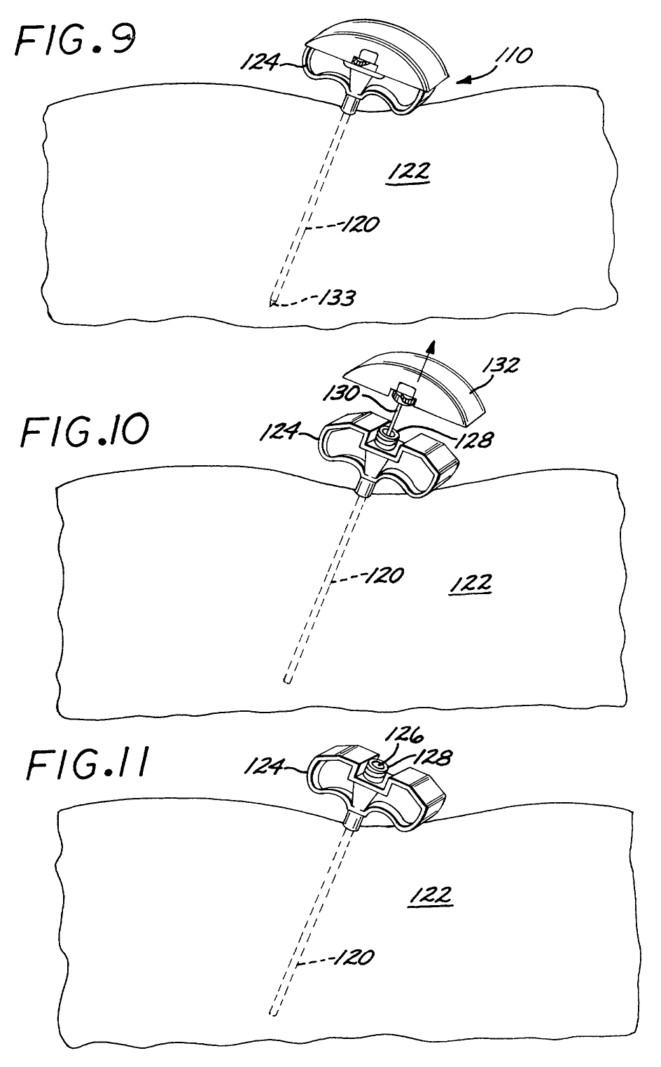

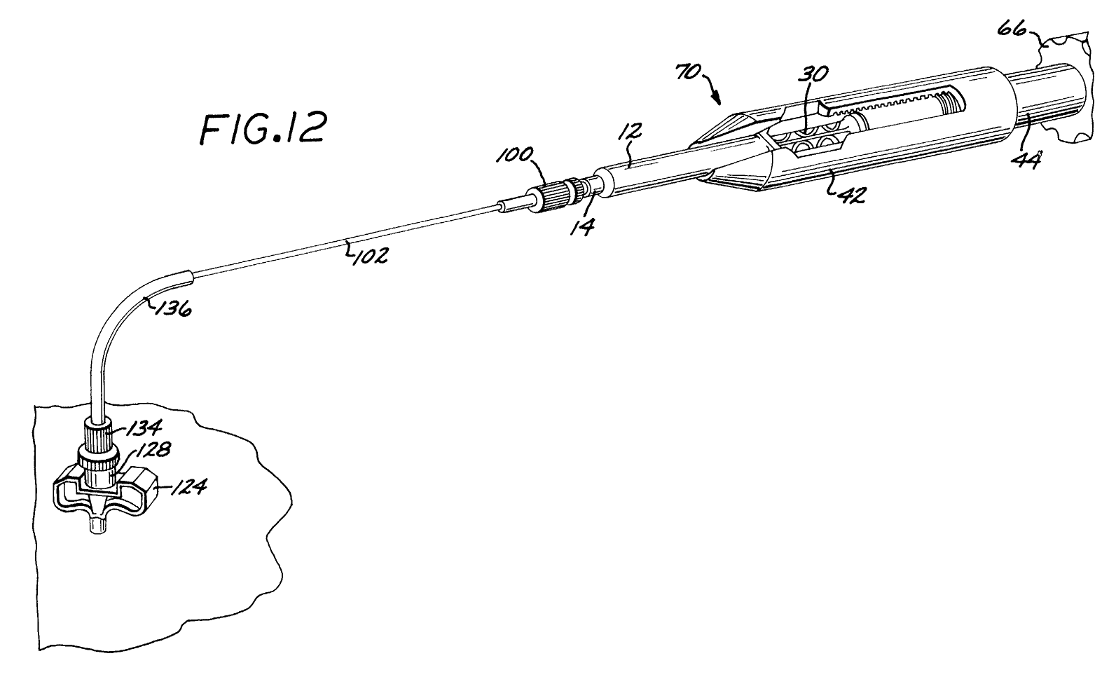

Referring now to the drawings, FIGS. 1, 4, and 5 illustrates a bone cement syringe 10, in accordance with a preferred embodiment of the invention. The syringe 10 comprises a barrel 12 that may be filled with a predetermined volume (typically, for example, 10 cc) of bone cement or other therapeutic liquid of relatively high viscosity. The distal end of the barrel 12 tapers down to a distal outlet tip 14, which may be internally threaded (at 16) for coupling to a conventional Luer fitting 100 (FIG. 12) at one end of a length of flexible tubing 102 (FIG. 12). The other end of the tubing 102 is typically coupled to a cannula 120 (FIGS. 9-11) for introducing the liquid expressed from the syringe 10 to a surgical site (such as a bone, in the case of bone cement) within a patient's body, as will be described in more detail below.

The barrel 12 has an open proximal end 20, and a flared peripheral flange or rim 22 around the proximal end. The rim 22 flares proximally and radially outwardly, creating a funnel for the proximal end opening. The flared peripheral rim 22 circumscribes less than 360° of the proximal end opening, in the range of about 210° to about 270°, and preferably about 240°. This creates an interruption or gap 24 in the funnel for direct access to the open proximal end of the barrel 12. For bone cement of the less viscous type, the funnel allows quick and accurate pouring of a measured amount of the bone cement into the syringe barrel 12 without spillage. For bone cement of the highly-viscous, paste-like type, the gap 24 in the funnel allows the proximal end opening to be directly accessed by a cement spatula or like instrument (not shown), so that the cement can be scraped off of the spatula into the barrel 12.

The syringe 10 has a plunger 30 that is installed for axial movement within the barrel 12 between a withdrawn position and an inserted position. The plunger 30 comprises a pair of flattened, elongate, longitudinal members 32 that intersect at right angles to form a shaft that is substantially cruciform in cross section (FIG. 8). The plunger 30 is reinforced by a truss element lattice 34 that connects the respective longitudinal members 32 of the plunger shaft. The proximal end of the plunger 30 is advantageously configured as a curved or radiused button or knob 36, while the distal end of the plunger 30 is attached to a piston 38 sized for a sliding frictional engagement against the interior wall surface of the barrel 12. The purposes and advantages of these structural features of the plunger 30 will be explained below.

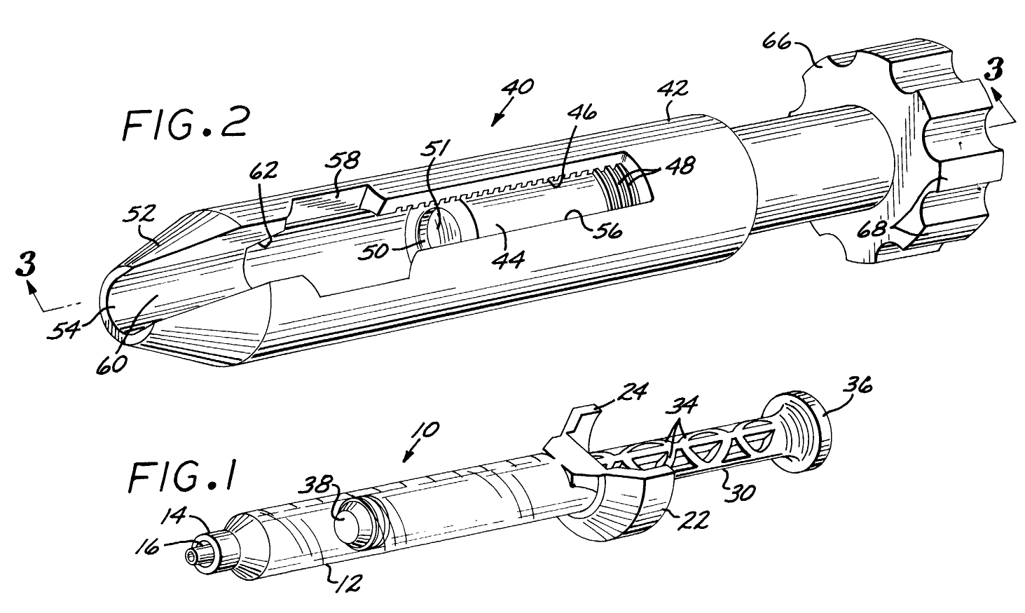

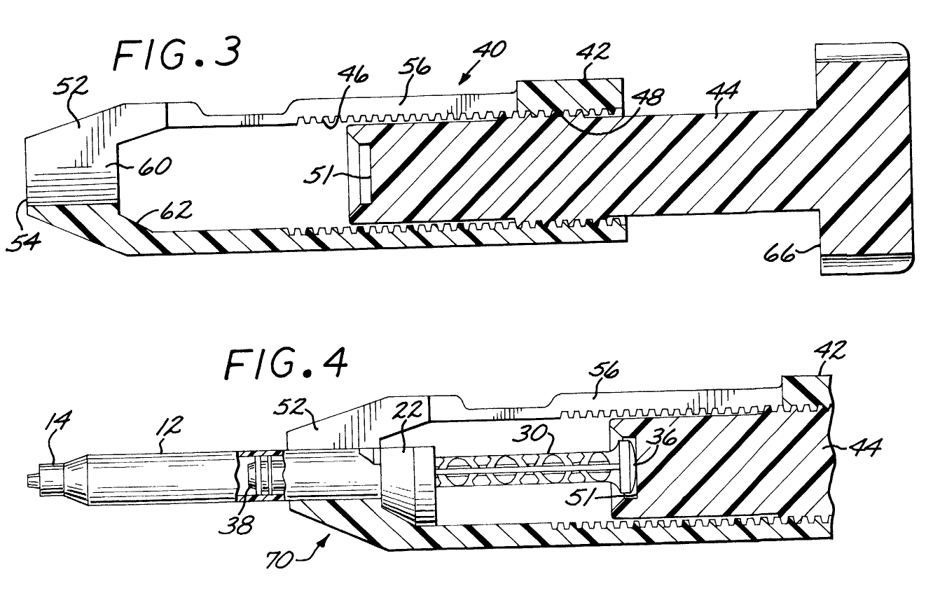

The syringe 10 is advantageously used in a syringe actuation device 40, best shown in FIGS. 2-5. The syringe actuation device 40 comprises a hollow tubular sleeve 42 and a substantially cylindrical driving element 44 that is dimensioned to fit within the sleeve 42. The sleeve 42 has an open proximal end and internal threads 46, while the driving element 44 has a portion with external threads 48 that mate with the internal threads 46 of the sleeve 42 when the driving element 44 is inserted into the proximal end of the sleeve 42. The driving element 44 has a distal end with a central recess 50 dimensioned to receive the proximal button or knob 36 of the plunger 30, and thereby defining a plunger seat 51.

The sleeve 42 has a tapered distal portion 52 terminating in a distal opening 54. A longitudinal slot 56 extends along most of the length of the sleeve 42 from the distal opening 54. The slot 56 is dimensioned for receiving a filled syringe 10 with its plunger 30 in its extended or withdrawn position, and it has a widened portion 58 to allow passage of the peripheral rim 22 of the syringe 10. The distal portion 52 defines a central bore 60 that terminates in the distal opening 54 and that is sized to hold, with a frictional fit, a syringe barrel 12 of the desired capacity (typically 10 cc). The tapered distal end 52 defines an internal shoulder 62 that is tapered at the same angle as the flare angle of the peripheral rim 22 of the syringe 10, thereby providing a seat for the rim 22, as shown in FIG. 4.

The driving element 44 has a proximal portion 66 that is advantageously of an enlarged diameter to provide a convenient hand grip. To this end, it may also be formed with longitudinal ridges 68 to provide a non-slip gripping surface.

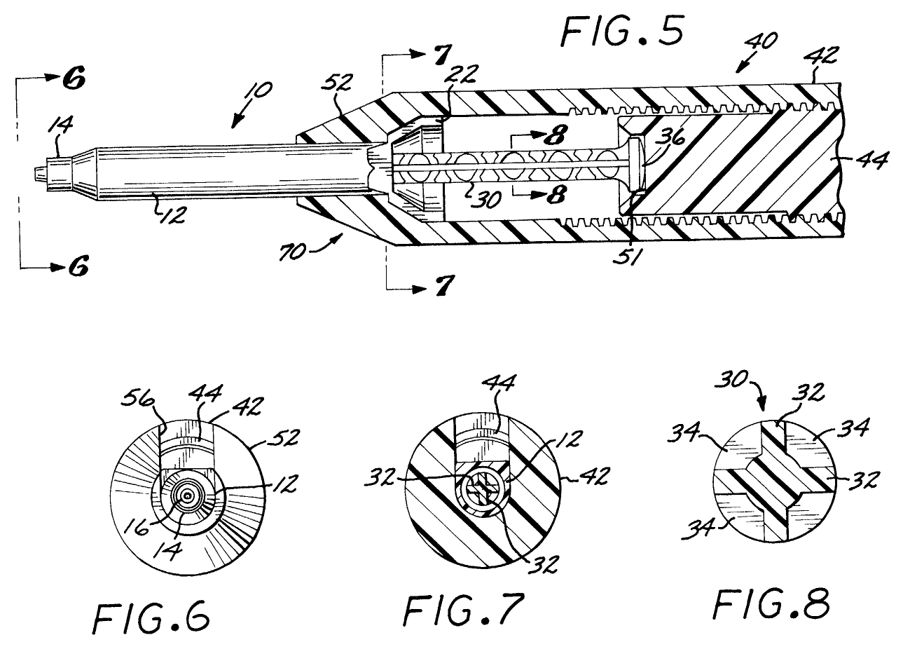

In use, the syringe 10, pre-filled with a measured volume of a liquid (such as bone cement) contained in the barrel 12, is installed within the sleeve 42 through the longitudinal slot 56. The syringe barrel 12 being filled, the plunger 30 is in its withdrawn (proximal) position, extending proximally from the proximal end of the barrel 12. The barrel 12 of the syringe 10 extends through the distal opening 54 of the sleeve 42, and it is held in place by the frictional fit of the barrel 12 in the bore 60, with the peripheral rim 22 of the syringe barrel seated against the tapered internal shoulder or seat 62. The driving element 44 may, at this point, be inserted into the proximal end of the sleeve 42 and threaded distally into the sleeve until the proximal knob or button 36 of the plunger 30 is received within the recess 50 in the distal end of the driving element 44 and is seated against the plunger seat 51. Thus, as shown in FIGS. 4 and 5, a bone cement delivery apparatus 70, comprising the syringe 10 and the actuation device 40, is ready for use to express the liquid contents of the barrel 12 out of the outlet tip 14 of the syringe 10, and to the surgical site through the tubing 102 and the cannula 120, as explained below.

FIGS. 9-11 illustrate the method of installation of a bone cement cannula 120 in a surgical site in a bone 122, using a cannula installation assembly 110, of a type that is commercially available from Manan Medical Products, of Wheeling, Ill. The cannula installation assembly 110 comprises a cannula 120 attached at its proximal end to the bottom or distal side of a grip 124. The grip 124 has a central passage 126 that extends from the proximal end of the cannula 120, through the body of the grip 124, and through a Luer-type fitting 128 on the proximal or top side of the grip 124. The cannula installation assembly 110 also comprises a bone needle 130 having a proximal end attached to and extending distally from the bottom or distal side of a needle handle 132, the latter being releasably attached to the fitting 128. When the needle handle 132 is attached to the grip 124, the needle 130 passes through the central passage 126 in the grip 124 and through the interior of the cannula 120, whereby the pointed tip 133 of the needle 130 is exposed through the distal end of the cannula 120 (FIG. 9). With the needle handle 132 thus attached to the grip 124, the needle 130 is driven into bone 122 to the desired site within the bone (FIG. 9). This locates the distal end of the cannula 120 at the desired surgical site within the bone 122. The needle handle 132 is then released from the grip 124 (FIG. 10), allowing the needle 130 to be withdrawn from the cannula 120, while leaving the cannula 120 in place (FIG. 11).

As shown in FIG. 12, a flexible tube or conduit 102 is connected to the grip fitting 128 by a mating Luer-type fitting 134 fixed to the distal end of the conduit 102. It may be advantageous to have the distal portion of the conduit 102 pass through a semi-rigid tubular stand-off 136 having a distal end fastened to the fitting 134 on the distal end of the conduit 102. The stand-off 136 creates a gentle bend in the distal portion of the conduit that allows the bone cement delivery apparatus 70 to be held in an optimum orientation relative to the cannula 120 without causing a potentially flow-impeding sharp bend or kink in the conduit 120.

To express the contents of the syringe 10, the driving element 44 is threaded further distally within the sleeve 42, thereby pushing the plunger 30 distally, toward its inserted position within the barrel 12, through the engagement between the plunger seat 51 and the button or knob 36 at the proximal end of the plunger 30. This process may be continued until the plunger 30 is in its fully inserted (distal) position, at which point the entire volume of liquid contained within the barrel 12 has been emptied therefrom. It will be appreciated that this process can be interrupted at any desired position(s) of the plunger 30 to express a part of the contents, or to express the contents in desired increments. The contents of the syringe 10 is then caused to flow to the surgical site in the bone 122 through the conduit 102 and the cannula 120.

It will be appreciated that the rotation of the driving element 44 as it is threaded into the sleeve will impart a torque or torsion to the plunger 30. This torque or torsion will be resisted by the frictional force between the liquid in the syringe barrel 12 and the interior walls of the syringe barrel. If the liquid is highly viscous, as is the case with many types of bone cement, these forces can place a torsional stress on the plunger 30 that could cause it to break. By having a radiused surface on the knob or button 36 at the proximal end of the plunger 30, the amount of torque transmitted to the plunger 30 by the driving element 44 is reduced. Furthermore, the truss element lattice 34 of the plunger shaft provides additional torsional rigidity and strength to minimize the likelihood of plunger breakage.

The screw mechanism action of the driving element 44 within the sleeve 42 provides a marked mechanical advantage that facilitates the dispensing of highly viscous liquids, such as bone cement, from the syringe 10. Furthermore, the partial or incremental dispensing of the syringe contents can be more easily controlled, by means of the screw mechanism, as compared with manually actuating the plunger by pressure applied directly by the user's thumb. Contributing to the control is the visibility of the entire length of the syringe barrel 12 through the longitudinal slot 56. In addition, the syringe actuation device 40 can easily be re-used. The empty syringe 10 can easily be removed and replaced with a new syringe.

While a preferred embodiment of the invention has been described herein, it will be appreciated that a number of modifications and variations will suggest themselves to those skilled in the pertinent arts. For example, the specific structure of the actuation device 40 described herein is exemplary only, and many alternative structures and configurations may suggest themselves. Such modifications, as well as others that may suggest themselves, are considered to be within the spirit and scope of the present invention, as defined in the claims that follow.

What is claimed is:

1. A syringe, of the type comprising a barrel having a distal outlet end and an open proximal end, and a plunger that is axially movable within the barrel, wherein the improvement comprises:
an outwardly and proximally flared peripheral rim around the proximal end of the barrel.

2. The syringe of claim 1, wherein the peripheral rim circumscribes less than 360° of the proximal end of the barrel, thereby defining a gap in the rim that provides direct access to the open proximal end of the barrel.

3. The syringe of claim 2, wherein the rim circumscribes between 210° and 270° of the proximal end of the barrel.

4. The syringe of claim 3, wherein the rim circumscribes about 240° of the proximal end of the barrel.

5. A syringe of the type comprising a barrel having a distal outlet end and an open proximal end, and a plunger movable axially within the barrel, the plunger having a shaft extending proximally from the proximal end of the barrel, wherein the improvement comprises:
a reinforcing structure incorporated in the shaft that provides increased torsional rigidity and improved dispersion of torsional loads.

6. The syringe of claim 5, wherein the shaft comprises a pair of flattened, elongate longitudinal members that intersect at right angles to form a shaft structure that is cruciform in cross section, and wherein the reinforcing structure comprises a truss element lattice formed between the longitudinal members.

7. The syringe of claim 5, wherein the plunger shaft has a proximal end, and wherein the improvement further comprises a radiused knob on the proximal end of the shaft.

8. A syringe of the type comprising a barrel having a distal outlet end and an open proximal end, and a plunger movable axially within the barrel, the plunger having a shaft extending proximally from the proximal end of the barrel, wherein the improvement comprises:
a reinforcing structure incorporated in the shaft that provides increased torsional rigidity and improved dispersion of torsional loads; and
an outwardly and proximally flared peripheral rim around the proximal end of the barrel.

9. The syringe of claim 8, wherein the peripheral rim circumscribes less than 360° of the proximal end of the barrel, thereby defining a gap in the rim that provides direct access to the open proximal end of the barrel.

10. The syringe of claim 9, wherein the rim circumscribes between 210° and 270° of the proximal end of the barrel.

11. The syringe of claim 10, wherein the rim circumscribes about 240° of the proximal end of the barrel.

12. The syringe of claim 8, wherein the shaft comprises a pair of flattened, elongate longitudinal members that intersect at right angles to form a shaft structure that is cruciform in cross section, and wherein the reinforcing structure comprises a truss element lattice formed between the longitudinal members.

13. The syringe of claim 12, wherein the plunger shaft has a proximal end, and wherein the improvement further comprises a radiused knob on the proximal end of the shaft.
